(19) United States
(12) Patent Application Publication
(10) Pub. No.: US 2009/0035190 A1
(43) Pub. Date: Feb. 5, 2009

(54) Sports Figure Air Freshener With Movable Ball

(76) Inventor: Omri Mutsafi, Dallas, TX (US)

Correspondence Address: Locke Lord Bissell & Liddell LLP;Attn: Michael Ritchie, Docketing, 2200 Ross Avenue, Suite # 2200, DALLAS, TX, 75201-6776, US

(21) Appl. No.: 12/168,960

(22) Filed: Jul. 8, 2008

Related U.S. Application Data
(60) Provisional application No. 60/935,253, filed on Aug. 2, 2007.

(51) Int. Cl. A61L 9/12

(52) U.S. Cl. 422/123

(57) ABSTRACT

The present invention provides for an air freshener in the shape of a sports figure that has a sports ball-shaped piece attached via a flexible strip that moves freely towards and away from the sports figure as the air-freshener moves. The air-freshener comprises a sports figure-shaped air freshener, a ball-shaped piece, a hanger for hanging the air freshener from a rearview mirror and a flexible strip that attaches the ball-shaped piece to the hanger. In one preferred embodiment, the air freshener depicts a football player, and the ball-shaped piece is shaped like a football. In another preferred embodiment, the air freshener depicts a soccer player, and the ball-shaped piece is shaped like a soccer ball. As the air freshener moves, the flexible strip moves and the ball moves towards and away from the sports figure. The flexible strip is of an appropriate length so that the ball-shaped piece contacts the foot of the soccer player or the hands of the football player when the flexible strip has moved the ball-shaped piece adjacent to the body of the sports figure.

### RELATED APPLICATION

This application claims the benefit of U.S. Provisional patent application Ser. No. 60/935,253 filed Aug. 2, 2007.

### BACKGROUND OF THE INVENTION

1. Field of the Invention

This invention relates generally to an air freshener in the shape of a sports figure and more particularly to a sports figure air freshener that has a sports ball-shaped piece attached via a flexible strip that moves freely towards and away from the sports figure.

2. Description of Related Art

Air fresheners are commonly used in automobiles and homes to help freshen or scent musty or stagnant air. There are several types of air fresheners including ones made of scented paper or card stock as well as plastic air fresheners that have reservoirs of beads, wax or oils. Paper air fresheners are commonly used in automobiles, and most are configured to hang from the car's rearview mirror. Automobile paper air fresheners have a wide variety of shapes, sizes and colors, and air fresheners depicting sports figures are known in the art. These simple air fresheners have no moving parts.

There are air fresheners for automobiles that are formed in the shape of sports figures that have movable parts, such as disclosed in U.S. Pat. No. 6,764,656 to Matulevich. The air freshener disclosed in Matulevich is shaped like a football player and is designed to clip to one of the automobile's air-conditioning vents. The arms of the football player, one of which is holding a football, are pinned to the center of the body and are free to rotate vertically around the body. As the air from the vent passes the air freshener, the arms rotate giving a rudimentary appearance of the player “throwing” the ball. In this design, the ball never moves away from the player's hand, and the player's arms move in a nonrealistic circular motion. Furthermore, the arms move only when air is flowing from the automobile's air vents. Also, the air freshener is not easily visible to individuals outside of the car because it is attached low in the car, below the windows on the air-conditioning vents.

It is therefore desirable to have an air freshener that is easily visible to others and movable in a life-like manner, wherein the movement is also not dependent on whether the air-venting system of the automobile is operating

### SUMMARY OF THE INVENTION

The present invention provides an air freshener in the shape of a sports figure that has a sports ball-shaped piece attached via a flexible strip that moves freely towards and away from the sports figure. The air-freshener of the present invention comprises a sports figure-shaped air freshener, a ball-shaped piece, a hanger for hanging the air freshener from a rearview mirror and a flexible strip that attaches the ball-shaped piece to the hanger or to the sports figure. In one preferred embodiment, the air freshener depicts a football player, and the ball-shaped piece is shaped like a football. In another preferred embodiment, the air freshener depicts a soccer player, and the ball-shaped piece is shaped like a soccer ball.

The air freshener component of the invention consists of a sports player-shaped body. The body could depict any type of sports player and can be designed to match the uniform of any team or any particular player or it could be designed to be a generic player. The shape of the ball and the coloring will of course depend on the type of sport depicted by the sports player (e.g., a football player freshener has a football, a soccer player freshener has a soccer ball, etc.). The hanger is attached to the top of the body of the air freshener and is designed such that the hanger cooperates and fits with any standard rearview mirror in any car. The flexible strip has the ball-shaped piece attached to one end, and the other end is attached to the hanger of the air freshener.

The flexible strip affords movement. As the air freshener experiences movement, either by the movement associated with an automobile going over a bump or turning or by someone just holding and moving the air freshener, the flexible strip flexes and bends and the ball moves toward and away from the air freshener. The flexible strip is sized lengthwise such that when the flexible strip is bent and the ball is adjacent to the air freshener, the ball lines up with and contacts the air freshener in an exact desired location. For example, if the air freshener is a football player, then the flexible strip will be a length such that when the strip is bent, the football contacts the player's outstretched hands. Similarly, if the air freshener is a soccer player, then the flexible strip will be a length such that when the strip is bent, the soccer ball contacts the player's foot.

The novel features and construction of the present invention, as well as additional objects thereof, will be understood more fully from the following description when read in connection with the accompanying drawings.

### BRIEF DESCRIPTION OF THE DRAWINGS

The improved process of the invention is further described and explained in relation to the following figures of the drawings wherein:

FIG. 1 is a side-view of a soccer player-shaped air freshener with a movable soccer ball; and

FIG. 2 is a side-view of a football player-shaped air freshener with a movable football.

Like reference numerals are used to describe like parts in all figures of the drawings.

### DESCRIPTION OF THE PREFERRED EMBODIMENTS

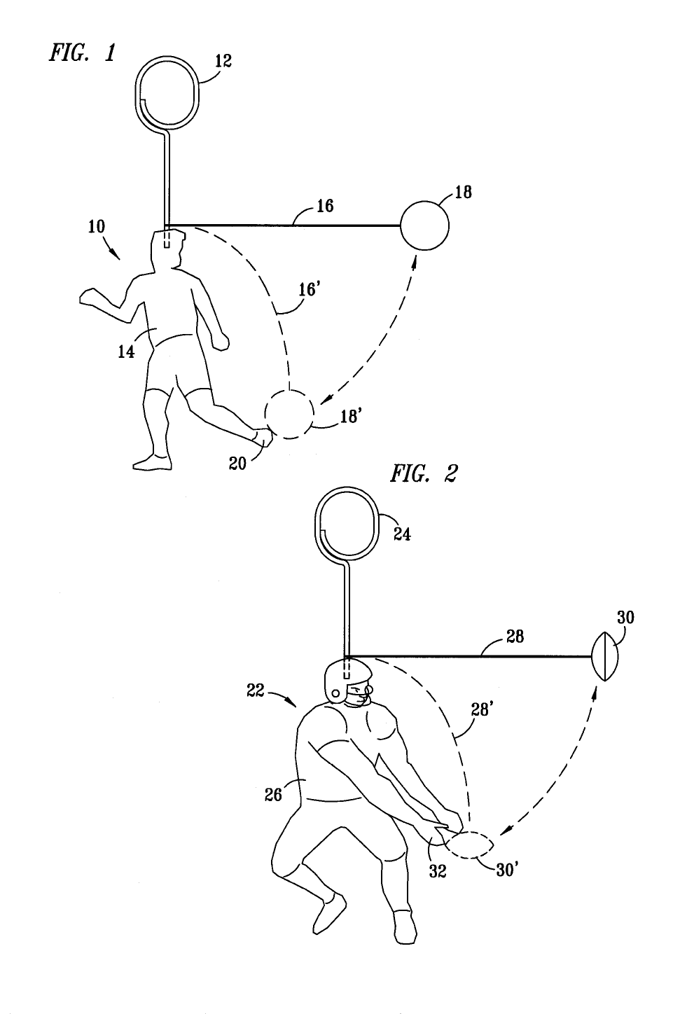

Referring to FIG. 1, a preferred embodiment of air freshener 10 is shown in the shape of a soccer player. Body 14 of air freshener 10 (as well as body 26 of air freshener 22 in the shape of a football player as shown in FIG. 2) may be constructed of any suitable air freshener material including paper or paper board, plastic or any other material suitable for shaping a sports figure. Air freshener 10 can use any of the known air freshening techniques. For example, aromatic agents such as beads, waxes or oils in an absorbent material could be incorporated into a paper version of air freshener 10 A hidden reservoir of scented agent could be used in conjunction with a plastic version of air freshener 10. Body 14 can be designed in any shape of any sports figure and can be colored to any variation of colors. In a preferred embodiment, body 14 would be designed, shaped and/or colored to match the uniforms of professional sports teams. Thus, the air freshener would appeal to the fans of that particular professional sports team.

Air freshener 10 is comprised of hanger 12, body 14, flexible strip 16 and ball-shaped piece 18. Hanger 12 is designed to allow air freshener 10 to hang from a standard rearview mirror in any automobile. Hanger 12 can be made from any suitable material, such as metal, elastic materials or plastic, and can have any number of designs as long as it is able to hang from a rearview mirror. Hanger 12 could also be a looped piece of elastic material, such as a string or rubber. Hanger 12 is attached to body 14 of air freshener 10. In the preferred embodiment, Hanger 12 is made of plastic with a loop at the top to fit over the stem of a standard car rearview mirror.

Body 14 is designed so as to resemble a sports figure in an action pose. In a preferred embodiment illustrated in FIG. 1, body 14 is shown in a kicking position. Ball-shaped piece 18 is shaped like a soccer ball in FIG. 1 and could be manufactured out of any number of materials including paper, paper board, plastic or metal. Ball-shaped piece 18 is attached to air freshener 10 via flexible strip 16. Flexible strip 16 is spring-like in that it freely moves. Flexible strip 16 is capable of moving, and has a free range of motion between the position shown by a solid line in FIG. 1 and position 16′, which is shown by a dotted line. When flexible strip 16 is in position 16′, ball-shaped piece 18 has moved to position 18′. In position 18′, ball-shaped piece 18 is adjacent to or touching foot 20 of soccer player shaped body 14. As flexible strip 16 moves back and forth as air freshener 10 is moved, the illusion is given that foot 20 of body 14 is kicking ball-shaped piece 18. Flexible strip 16 is designed to be a length such that when in position 16′, ball-shaped piece 18 is in position 18′ and adjacent to foot 20. Flexible strip 16 is attached to either hanger 12 or any portion of body 14 as long as flexible strip 16 can move ball-shaped piece 18 towards and away from the body to give a realistic sports movement. Flexible strip 16 is preferably made of a thin strip of metal, but could conceivably be comprised of any other flexible material that allows for a free range of movement.

Referring to FIG. 2, an additional preferred embodiment of air freshener 22 is shown, which is functionally identical to air freshener 10 from FIG. 1. Air freshener 22 has body 26 in the shape of a football player with outstretched hands 32. Hanger 24 is attached to body 26 and allows air freshener 22 to be hung from a rearview mirror. Flexible strip 28 connects ball-shaped piece 30 to body 26. Ball-shaped piece 30 is shaped like a football because body 26 is shaped like a football player. Flexible strip 28 moves to position 28′, which moves ball-shaped piece 30 to position 30′. Position 30′ is adjacent to and touching outstretched hands 32 of body 26, which gives the illusion that body 26 is catching ball-shaped piece 30 as flexible strip 28 moves ball-shaped piece 30 away from and towards hands 32.

Two preferred embodiments are shown in the figures, but any number of sports-shaped bodies could be used as long as a ball-shaped piece would move towards and away from a sports-shaped figure. For example, a basketball player could be shooting a basketball, a baseball player could be hitting a ball, or a golfer could be hitting a golf ball. Preferably these air fresheners are for use in automobiles because the natural motion of the automobile will cause the flexible strip to move the ball-shaped piece towards and away from the player. However, these air fresheners could also be used in homes or offices as well and a person could easily move the air freshener to cause the ball-shaped piece to move.

The utility of the movement of a ball-shaped piece due to a flexible strip as described herein is not limited to air fresheners. The cardboard piece in the shape of a sportsman could be a plain piece of cardboard or plastic and not an air freshener. This would appeal to persons who are interested in a moving decoration for their automobile or office, but who are disinterested in having an air-freshener.

Other alterations and modifications of the invention will likewise become apparent to those of ordinary skill in the art upon reading the present disclosure, and it is intended that the scope of the invention disclosed herein be limited only by the broadest interpretation of the appended claims to which the inventors are legally entitled.

1. An air freshener comprising:
a body;
a piece that is not part of the body; and
a flexible strip that extends between the body and the piece;
wherein as the flexible strip flexes downward, the piece moves towards the body, and as the flexible strip then returns upward, the piece moves away from the body.

2. The air freshener of claim 1 further comprising a hanger attached to the body to allow the air freshener to hang.

3. The air freshener of claim 1 wherein the body depicts a sports figure and the piece is the shape of a ball that corresponds to the sport depicted by the body.

4. The air freshener of claim 3 wherein the body depicts a football player and the piece is shaped like a football.

5. The air freshener of claim 4 wherein the body further comprises a set of hands, and the flexible strip is of a length that when fully flexed downward, the piece is adjacent to the hands of the body, which gives the appearance of the body catching the football-shaped piece.

6. The air freshener of claim 3 wherein the body depicts a soccer player and the piece is shaped like a soccer ball.

7. The air freshener of claim 6 wherein the body further comprises a foot, and the flexible strip is of a length that when fully flexed downward, the piece is adjacent to the foot of the body, which gives the appearance of the body kicking the soccer ball-shaped piece.

8. The air freshener of claim 1 wherein the body is paper.

9. The air freshener of claim 1 wherein the body is plastic.

10. The air freshener of claim 1 wherein the piece is paper.

11. The air freshener of claim 1 wherein the piece is plastic.

12. The air freshener of claim 1 wherein the flexible strip is metal.

13. A device comprising:
a body;
a piece that is not part of the body; and
a flexible strip that extends between the body and the piece;
wherein as the flexible strip flexes downward, the piece moves towards the body, and as the flexible strip then returns upward, the piece moves away from the body.

14. The device of claim 13 further comprising a hanger attached to the body to allow the air freshener to hang.

15. The device of claim 13 wherein the body depicts a sports figure and the piece is the shape of a ball that corresponds to the sport depicted by the body.
